(19) United States
(12) Patent Application Publication
(10) Pub. No.: US 2023/0165822 A1
(43) Pub. Date: Jun. 1, 2023

(54) URINARY ALKALIZING MEDICINAL AND/OR PHARMACEUTICAL COMPOSITION FOR THE ORAL TREATMENT OF INTERSTITIAL CYSTITIS / BLADDER PAIN SYNDROME (IC/BPS) AND FORMULATION THEREOF

(71) Applicants: Sándor LOVÁSZ, Budapest (HU); Gábor RÉNYI, Budapest (HU); János GIBER, Budapest (HU); Péter BIRINYI, Budapest (HU)

(72) Inventors: Sándor LOVÁSZ, Budapest (HU); Gábor RÉNYI, Budapest (HU); János GIBER, Budapest (HU); Péter BIRINYI, Budapest (HU)

(21) Appl. No.: 17/922,221

(22) PCT Filed: Apr. 29, 2021
(86) PCT No.: PCT/HU2021/000004

§ 371 Date: Oct. 28, 2022

(30) Foreign Application Priority Data
Apr. 29, 2020 (HU) P2000144

(51) Int. Cl. A61K 31/194; A61K 9/20

(52) U.S. Cl. CPC A61K 31/194; A61K 9/2054; A61K 9/2009; A61K 9/2013; A61K 9/2095

(57) ABSTRACT

The present invention relates to novel medicinal and/or pharmaceutical urinary, extended-release alkalizing composition advantageously a tablet for the oral treatment of interstitial cystitis/bladder pain syndrome (IC/BPS) and/or for general alkalization of the human body for long term where according to the subject matter of the invention the composition is potassium free, the composition has a sustained and/or controlled release dosage form and comprises following components advantageously with certain consistence: Citric acid, Sodium citrate, Magnesium citrate, and comprises following components advantageously with a certain consistence as excipients: Aerosil, Avicel DG, Benecel hypromellose microcrystalline cellulose (HPMC) as grade, advantageously Benecel K100M PH DC HPMC and Magnesium stearate. The subject matter of the present invention relates furthermore to novel medicinal and/or pharmaceutical urinary, extended-release alkalizing composition wherein a single administered dose of the composition achieves a therapeutic alkalizing concentration for providing a neutral pH value of the urine between values of 6.9 to 7.5 advantageously 7.38 in an individual for 12 hours. According to the release kinetics of the active ingredient of the composition the final conclusion is that the sustained and/or controlled release tablet according to the invention provides in vivo constant and continuous therapeutic concentration in case of 2×1 daily dosage. The subject matter of the present invention furthermore relates to the process for the formulation of the composition according to the invention because during the homogenization of the above described active ingredients a special formulation process is needed for avoiding liquefaction and the formation of eutectic.

The present invention relates to novel medicinal and/or pharmaceutical urinary, extended-release alkalizing composition advantageously a tablet for the oral treatment of interstitial cystitis/bladder pain syndrome (IC/BPS) by alkalizing the urine and/or for general alkalization of the human body for long term where according to the subject matter of the invention the composition is potassium free, the composition has a sustained and/or controlled release dosage form and comprises following components advantageously with the following consistence as active ingredients in weight percentage:

Citric acid, advantageously 50 to 100 mg, especially advantageously 68.00 mg or 53.17 mg and
Sodium citrate, advantageously 130 to 250 mg, especially advantageously 183.00 mg or 143.09 mg and
Magnesium citrate, advantageously 190 to 290 mg, especially advantageously 243.00 mg or 190 mg;
and comprises following components advantageously with the following consistence as excipients in weight percentage where the total weight percentage of the above active ingredients in the especially advantageous case is totally 60% and this percentage depends on the advantageous ranges of the excipients:
Aerosil, advantageously 0.5% weight percentage;
Avicel DG, advantageously 15% to 25%, especially advantageously 19% weight percentage;
Benecel hypromellose microcrystalline cellulose (HPMC) as grade, advantageously Benecel K100M PH DC HPMC advantageously 15% to 25%, especially advantageously 20% weight percentage, where the number (K100) indicates the viscosity of the grades, the PH means pharmaceutical quality and DC indicates that the grade can be pressed;
Magnesium stearate, advantageously 0.5% weight percentage.

The subject matter of the present invention relates furthermore to novel medicinal and/or pharmaceutical urinary, extended-release alkalizing composition wherein a single administered dose of the composition achieves a therapeutic alkalizing concentration for providing a neutral pH value of the urine between values of 6.9 to 7.5 advantageously 7.38 in an individual for about 8 to about 14 hours advantageously 12 hours.

The subject matter of the present invention furthermore relates to the process for the formulation of the medicinal and/or pharmaceutical urinary, extended-release alkalizing composition according to the invention comprising the above-described components in the especially advantageous amounts as described above by in a solid form advantageously a tablet by the following steps:

during the homogenization of the above described active ingredients a special formulation process is needed for avoiding liquefaction and the formation of eutectic therefore
each active ingredient was pulverized separately till reaching the particle size of 6 fine-mesh sieve;
afterwards the excipient Aerosil with moisture retention capability was added to each active ingredient separately in small parts by continuous mixing in an amount of totally 0.5%;
afterwards the optimization of the rheological features of granulates for formulating tablets was provided by adding excipient Avicel DG with high moisture retention capability to each active ingredient separately in small parts by continuous mixing in an amount of totally 19%;
afterwards for providing the controlled release feature, Benecel K100M PH DC HPMC grade was added to each active ingredients separately in an amount of totally 19% and each of the mixture of the components were homogenized separately.
after combining and homogenization of the three above prepared mixture of the excipients and ingredients the required lubricant effect was provided by adding excipient Magnesium stearate in an amount of 0.5%.
The granulates prepared by dry granulation were formulated to tablets by an excenter type Korsch tablet press machine.

### HISTORY, THE STATE OF THE ART

According to the definition of the National Institute of Diabetes and Digestive and Kidney Diseases (NIDDK, the USA) Interstitial Cystitis/Bladder Pain Syndrome (henceforth IC/BPS) is a chronic, or long lasting, condition that causes painful urinary symptoms.¹ Its symptoms greatly affect the patients' quality of life.² As IC/BPS progresses, the pain and the frequent voiding (which may exceed more than 80 occasions per day) can severely impede work, sexual intercourse, social life and night-time resting. Other chronic conditions occur more frequently in IC/BPS patients than in the general population.³

According to our present knowledge, there is no permanent cure for IC/BPS.⁴ On the other hand, patients can be free from symptoms for years, and their normal quality of life can be preserved, assuming they get the appropriate treatment. Due to the increasing number of diagnosed cases and the length of treatment, IC/BPS shall demand an increasing amount of resources from the healthcare systems in the near future.

IC/BPS: The Known Facts

The causes of IC/BPS are still not known. The possible explanations are the dysfunction of the related nerves, autoimmune problems, allergic reactions, and stress. Hereditary factors may take part, too. Nevertheless, none of these hypotheses have been proven scientifically.

The condition itself, on the other hand, has been well described.⁵ The symptoms occur because of the inadequate status of the mucosa of the bladder and the upper part of the urethra. The healthy layer of mucosa—which consists of glucose amino glycan or GAG—prevents salts, acids and other urinary products (which are present in the urine naturally) interfering with the deeper layers of this tissue; most importantly with the sub-mucosal pain receptors. In the case of IC/BPS, this GAG-layer is being damaged and enables the compounds described above to reach the tissues. This process results in a sterile inflammation—in which there are no bacteria present. The inflammation can spread to the deeper layers of the bladder wall, too, and leads to an increased amount of mast cells. These cells secrete histamine, which increases the pain. The constant irritation raises the number of pain receptors, which makes the symptoms worse. If the inflammation persists for years, other elements of the connective tissues build up in the edematous tissue, which makes the bladder wall lose its elastic properties. At the end of this process, the bladder turns into a shrunken bladder (a hard bladder with very low capacity), which is an irreversible condition. As a consequence, kidney failure may appear, too.

Since it, the cause of the GAG-layer loss is not known, it is impossible to prevent IC/BPS. Moreover, there is no therapy available which cures the condition for good. The early diagnosis and the proper treatment can stop the progression of IC/BPS; therefore, in sense of the way, its effects are somewhat preventive. Controlling the urine pH is a vital part of the treatment, because not only the concentrated but also the too acidic—or even too alkalic—pH makes the symptoms more severe.

Diagnosis and Prevalence: Double Difficulty

Despite a lot of efforts taken to find any marker, so far nothing has been discovered that can be inarguably associated with IC/BPS.⁶ There are no alterations either that would refer to IC/BPS, without doubt, so using the most known imaging methods in themselves do not provide an unambiguous diagnosis. The image of the healthy bladder and the disrupted one may be identical. On the other hand, the insufficiency of the GAG-layer can refer to other diseases, too. Excluding malign transformations and infections is a necessary step, but even the presence of any other condition cannot rule out IC/PBS. Therefore, IC/BPS can sometimes only be diagnosed after the successful treatment of the easily identifiable condition.

The usual symptoms of IC/BPS can be divided into two major groups.⁷

Pain
Not only the urethra and the bladder can be affected, but also the lower abdomen, the pelvic or perineal area (moreover, in women the vagina, in men the scrota and the penis)
Its intensity may correlate with the filling of the bladder, whereas voiding may reduce it
Assuming the urethra is affected, sexual intercourse may be painful
Its amount varies from mild discomfort to severe, excruciating pain
In the beginning, the sparse and short painful periods are separated with long, symptomless intervals. As IC/BPS progresses, pain becomes permanent, and it can occur without any correlation to voiding. Patients may experience flare-ups, too.

Voiding
In the beginning, the frequency is slightly higher than normal. In severe cases 60-80 occasions a day is possible, too
Sudden urgency may occur, followed by spasms and pain
In mild cases, the abnormal frequency of voiding shows up only in daytime. With progression nocturia develops, the need for voiding can occur several times at night.
The amount of urine per voiding (the urine portion) is very small and correlates to the amount of liquid consumed.
In severe cases, the need for voiding persists after urinating too.

The presence of these symptoms varies by patients and affected by several factors. Namely, consuming certain foods and drinks, the amount of physical and/or mental stress, digestive disorders, urinary infections (UTIs) and (in women) their menstrual cycle (the symptoms are usually worse after ovulation).

Most urologists define a condition as IC/BPS if the characteristic symptoms persist for a certain period (1.5-6 months) given that every disease of similar symptoms can be excluded. Filling out questionnaires can identify the presence of symptoms; the O'Leary-Sant Symptom Index is one of the most frequently used ones⁸. However, because no lab tests or any other kind of examination can unequivocally confirm IC/BPS, the condition can never be diagnosed with a 100% certainty. Fortunately, not only are there a handful of supplemental examinations which can be used for refining the diagnosis, but also the medical practice has improved significantly in this field in recent years.

The occurrence of disease can usually be described by two kinds of data. Incidence means the newly registered cases during a certain period (usually a year). Prevalence, on the other hand, means the total amount of people affected by the disease at a certain point of time. In the case of IC/BPS, which appears to be a life-long condition, the latter data is relevant.

The international estimations of prevalence are based on the presence of symptoms, filling in questionnaires, and data on patients having diagnosed with IC/BPS. The number of people affected by IC/BPS is usually referred to as 100,000 people.

However, neither the questionnaires nor the way of their evaluation is standardized. Certain studies that used only the data gathered from doctors focusing on the diagnosed IC/BPS cases concluded a prevalence of 45-197/100,000.⁹ On the other hand, a survey in which households had been contacted by phone estimated 1,900-4,200/100,000 men and 2,750-6350/100,000 women affected by IC/BPS. A mere 10% of the latter group had been diagnosed.^{10 11} According to another research based on self-reporting via e-mail, IC/BPS can affect 258-13,114/100,000 people, depending on the way of calculations.¹²

In 2017 Interstitial Cystitis Association (ICA) reported that alone in the USA, there are 3-8 million women and 1-4 million men affected by IC/BPS.¹³ In recent years, this estimation seems to have been accepted by many relevant papers and organizations.¹⁴, ¹⁵ Considering the mean of both values, a prevalence of 2,400/100,000 appears to be a reasonable calculation.

Certain reports explain that in case of a percentage of the patients, the symptoms can disappear without any treatment (given that the condition has never been severe). Including this in our calculations, a prevalence of 2,000/100,000 must be close to the reality.

That said, the diagnosis rate of IC/BPS is less than 5-10%, even in the countries with the most advanced healthcare. There is no other disorder of this seriousness, which has a lower diagnostic rate. The disease is unknown to most people and a certain part of the medical society, too.

The healthcare sector needs to be enlightened, especially urologists, gynecologists, and family doctors since these are the groups most probably turned to by patients in the first place. It is also essential to give relevant information to the society about IC/BPS. The existence of a severe, painful disease like this, which can be fatal if remains untreated, must be a well-known fact.

Nevertheless, enlightening seems to be an ongoing trend, regardless of our effort.

As a consequence of the improvement of the diagnostic methods, the number of resources spent on the treatment of IC/BPS shall show an increasing trend worldwide.

The Treatment of IC/BPS and the Role of Alkalization Therein

Most guidelines—including the one of American Urological Association (AUA)—shares the view that the doctor should start with the least invasive method and progress step by step towards the more invasive technics.¹⁶

The least invasive therapeutic possibilities describe lifestyle changes. Diet has a major impact on the symptoms. IC/BPS food and drink lists are widely available on the internet¹⁷, ¹⁸, ¹⁹, and scientific papers have been published about this topic, too²⁰, ²¹. Most of the references agree that certain nourishments irritate the damaged bladder wall. Lists usually mention the following things:—
Caffeinated beverages
Alcoholic drinks
Hot and spicy foods
Sour and acidic foods, including carbonated drinks
Some fruit of high acid content
Tea or certain dietary supplements containing fragrance oil and/or volatile oil compounds
Herbal products

Not only are there nourishment categories on these lists which are increasing the acidity of urine but also most dietary recommendations are suggesting the alkalization of the urine, too.

If there is no improvement experienced, the next major line of treatment is oral therapy. The most common active ingredients are antihistaminic, non-steroidal or corticosteroid anti-inflammatories, tricyclic antidepressants, gabapentin nerve pain relieve, and pentosan-polysulfate sodium (PPS).

Urine alkalizing agents are regularly administered as well.

The correlation between the urine pH and the symptoms was discovered long ago; it has been described in several papers²²; recent guidelines usually mention it as an effective way to mitigate the symptoms.²³

If the oral therapy turns out to be ineffective, the next possibility is the local treatment: the GAG-layer replenishment substances are being instilled directly into the bladder. The approved drugs are different depending on the country. The most widespread substances are heparin, hyaluronic acid, chondroitin sulfate, pentosan polysulfate sodium, dimethyl sulfoxide (DMSO) and lidocaine. It is worth pointing out that alkalization is beneficial in this phase of the treatment, too; alkalized lidocaine is often used as an ingredient of the medicines (aka. bladder cocktails).²⁴ All the less invasive methods are usually performed during the local therapy.

Alkalization and dilution of the urine are essential for the proper treatment in all but the most severe cases.

Keeping the urine pH in the optimal range is crucial, regardless of the guideline being used for different other treatments of IC/BPS.

Alkalization overview Alkalization has been a part of the medical practice for a long time. There are several conditions in whose treatment alkalization plays a key role. The most important—and most common—are to mention are gout, kidney, and bladder stones. Due to the relatively high prevalence of these conditions, there are plenty of alkalizing medicines, medicinal compositions, dietary supplements, and other products on the market.

Their popularity can be demonstrated with the fact that Milurit (allopurinol) was the eighth most frequently prescribed medicine in Hungary in 2018 (more than 500,000 cases).²⁵ Its active agent, allopurinol, is used mostly for treating gout, however it is administered in case of other conditions, too, because it inhibits the synthesis of uric acid.²⁶

Regarding urinary alkalization, the most common medical products used in the USA contain sodium bicarbonate or citric acid/citrate salts as active pharmaceutical ingredients.²⁷ These products are available in every country, manufactured by different companies, under different names. Neut and Brioschi, brands from the USA, Sellymin from Canada, in some other countries simply active ingredient “sodium bicarbonate” is named (as a product). Alkurin, Cytra-K, Bicitra, and Virtrate products contain citric acid and different citric salts. Some of these products are for systematic alkalization, too.

Although the market seems to be diverse and overbought, it must be pointed out that none of these products have IC/BPS as their primary indication. There is one single alkalizing dietary supplement which is directly recommended in case of IC/BPS. It is named Prelief, and it works differently. Prelief does not alkalize the urine; it has to be taken with the food or beverage consumed so that it can reduce the acid content of the nourishment. In spite of the difference, Prelief appears to be the closest competitor of the composition according to the subject matter of the invention.

It is worth mentioning, though, that Prelief does not change the urine pH directly, so any factors that affect the acidity of the urine and are independent of nutrition still prevail.

A considerable portion of urinary alkalizing products can and should not be used for treating IC/BPS at all.

Firstly, certain products contain potassium, which is proven to make both the urinary and the pain syndromes worse, given that the GAG-layer of the bladder is already damaged.²⁸, ²⁹, ³⁰ Potassium, on the other hand, causes no problems in case of a healthy GAG-layer. Potassium citrate is generally used for treating gout since it brings the uric acid causing joint problems into a water-soluble form. Too much potassium, nevertheless, can lead to hyperkalemia, whose typical symptoms can be found in many of the leaflets of the alkalizing medicines, as notable side effects.³¹

Also, the increased potassium intake results in higher concentration of potassium in the bladder, which is seriously irritative.

Secondly, urine pH has a natural fluctuation. It happens due to several factors: food intake, exercise, daily routine, hormonal activities. This fluctuation cannot be felt in case of a healthy GAG-layer but can lead to flare-ups (sudden, short but severe periods of pain) in many of the IC/BPS patients. There are plenty of reports and summaries explaining the correlation between certain foods and flare-ups.³² Not only the acidic urine pH can raise the frequency of these incidents, but also the rapid changes. According to several patients' reports the alkaline urine (pH>7.5) can cause just as severe pain as the highly acidic (pH<6.0) does. Currently available alkalizing tablets have to be taken three or four times a day; typically after a meal and sometimes before bedtime, too—or even more frequently.³³, ³⁴ The way the traditional alkalizing tablets are being administered lead inevitably to the sudden changes of the urine pH. After meals—which usually makes the urine more acidic—the pH does rise quickly (in 15 minutes to 1 hour) and may reach the optimal range to compensate the effect of the food intake, but may enter the dangerously alkalic range, too. An even bigger issue is that the effect of these tablets lasts for 1-4 hours, so the daily mean value of the urine pH still remains in the highly acidic range. Therefore, for treating IC/BPS, a urinary alkalizing tablet of a slower, longer effect would be much more efficient.

It is worth mentioning that there are certain medicines on the market which are able to raise the urine pH and have a long-lasting effect or slow-release forms. Diamox is one of these products.³⁵ This, and other medicines, containing acetazolamide as an active pharmaceutical ingredient, are designed for completely different indications. Even if 90% of acetazolamide is said to reach the bladder, these drugs affect several other organs and tissues, and the risk of experiencing side effects is relatively high, too. Therefore, using them at the treating of IC/BPS is not recommended.

Methazolamide is another agent which may have some alkalizing effect, but since only a fraction of the compound reaches the bladder, its efficiency for treating urinary conditions is questionable.³⁶

That said, currently, there is neither alkalizing medicine, medicinal composition nor dietary supplement available on the market which would be able to raise the urine pH with a long-lasting, slow-release effect. Thus, according to the prior art there is no optimal way to alkalize the urine for IC/BPS patients.

### TARGET AND SOLUTION ACCORDING TO THE INVENTION

Using the traditional urinary alkalizing medicines was proved to be effective for raising the urine pH, but as it was described in the previous chapter, potassium caused the worsening of the symptoms during the process, lowering their efficacy.

According to the literature for the alkalization of the urine the optimal solution is the use of different citric salts like in case of e.g. Magurlit or Blemaren-N but both medicines contain potassium salts.

Using NaHCO₃ for urine alkalization is not a good solution because the acid content of the stomach immediately neutralises the effect thereof.

The citric salts are optimal for alkalizing the urine but because of their quick half period the alkalizing effect passes quickly and the therefore to keep the pH value in the appropriate neutral range the dosage of the normal alkalizing tablets comprising citric salts could be 4-6 tablets or more a day which is very high.

Using magnesium and sodium salts of citric acid is also optimal because magnesium and sodium are completely eliminating from the human body within 24-48 hours so a danger of a cumulation is excluded.

Therefore the composition according to the subject matter of the invention
should contain citric acid and different citrate salts,
the composition should be potassium-free and
to avoid pH value fluctuation of the urine the solution should be a sustained and/or controlled release composition advantageously a tablet.

According to several in vitro tests, a duration of 12 hours is also possible concerning the alkalizing effect of the tablet.

During this time, the tablet will release the active ingredients sustained and linearly as a controlled-release tablet a determined, nearly constant quantity of the active ingredient was released.
Using the composition according to the invention the urine pH fluctuation of the IC/BPS will be attenuated.
The fact that the tablet has to be taken fewer times and it affects the pH for a prolonged time definitely leads to some attenuation. With or without a considerable fluctuation if the pH remains in the 6.5-7.5 range (thus, it does not make the symptoms worse), the composition according to the invention can inarguably be the optimal alkalizing tablet for the treatment of IC/BPS.
IC/BPS is not the only condition in which the GAG-layer is damaged. There are plenty of urinary tract infections (UTIs) with a symptom of a deficient GAG-layer and/or an abnormally low urine pH. In these cases the composition according to the invention might be administered for symptomatic treatment, as an analgesic drug. Also, long-lasting alkalization might be useful for certain other conditions, too, namely radiation cystitis or chemo-cystitis following BCG or cytostatic drug instillations.
Another disease which could be treated with the alkalizing composition according to the invention is gout. Gout is a common disease which is definitely worth focusing on. As it has been mentioned before, potassium seems to be useful for treating this condition; allopurinol inhibits the production of uric acid. The urine itself, regarding gout, is often stabilized around a pH value of 5. Raising this might help the effect of allopurinol by raising the urine pH, especially in cases, where potassium-containing drugs are contraindicated.

Based on the above concerning of the preparation of the optimal alkalizing agent for oral treatment of the IC/BPS by controlling the pH of the urine the target of the invention can be solved as follows:
1) The pH value of the urine should be provided for long term and continuously neutral and the optimal pH value of the urine should be between 6.9 and 7.5 advantageously 7.38.
2) The composition according to the invention should provide the required continuous neutral pH value of the urine for 24 hours by 2×1 daily oral dosage of a sustained or controlled release composition advantageously tablet where the active ingredient release during this period should be nearly constant.
3) The composition according to the invention should be potassium ion free, because the potassium is strongly irritating the urethra and bladder of the IC/BPS patients as it is well known from the literature. By using a potassium-free alkalizing tablet the pain and continuous micturition of the patient can be avoided.

Preparing a sustained, controlled release tablet with the above indication the optimal choice was using Benecel hypromellose microcrystalline cellulose (HPMC), advantageously Benecel K100M PH DC HPMC as grade, because polymers or Carbomer matrix systems cannot be used because of the high Mg²⁺ and Na⁺ content thereof.

The osmotic pressure generated by the cations namely would have destroyed the texture of the matrix.

The HPMC is much more resistant against the osmotic pressure generated by cations and has a high viscosity which is optimal for a formulation of a sustained, controlled release tablet.

Finding the exact optimal consistence of the controlled release alkalizing tablet was a very important target and the result was found only by careful and detailed investigation of the dissolution of the active ingredient by using HPLC (High Pressure Thin Layer Chromatography) technique.

There were features of two excipients investigated:
a) The active ingredient components of the composition according to the invention are extremely hygroscopic so because of the liquefaction the optimal quantity of the excipient Avicel DG with moisture retention capability should be examined.
b) Because of finding the optimal active ingredient release of the composition for 12 hours the optimal consistence and quantity of the HPMC grade component by investigating the dissolution, more precisely the dissolution efficacy of the active ingredient by HPLC.
c) The HPLC investigations were implemented depending on the above two parameters.

During the investigation of the active ingredient release following parameters were used:
37° C.
75 turn/minute
2.4 ml/sample
phosphate puffer: pH 6.8

Mathematical Analysis of the Drug Release Profiles in HPLC Investigations.

To compare the individual dissolution data of the two mostly varying 3.3 tablet compositions, similarity or difference factors were calculated, as a model independent approach. Dissolution efficacies were also calculated for the average dissolution data (1).

$$f 1 = \frac{{\sum }_{j = 1}^{n} \vert {R}_{j} - {T}_{j} \vert}{{\sum }_{j = 1}^{n} {R}_{j}} \times 100$$

where n is the sampling number, Rj and Tj are the percent dissolved of the reference and the test products at each time point j.

f2=50×log{[1+(1/n)Σⱼ₌₁ⁿwⱼ|Rⱼ−Tⱼ|²]^{-0.5}×100}

where wj is an optional weight factor.

$$DE = \frac{{\int }_{0}^{t} y \times dt}{{y}_{100} \times t} \times 100 \%$$

where y is the drug percent dissolved at time t.

For the determination of release kinetics of active ingredient, release data was fitted to zero-order, first-order and Korsmeyer-Peppas model equations in MS Excel.

$$\begin{matrix}Q = {Q}_{0} + {k}_{0} t \\ {Q}_{t} = {Q}_{0} \times {e}^{- {k}_{1} t} \\ \frac{{Q}_{t}}{{Q}_{∞}} = {k}_{k p} {t}^{n}\end{matrix}$$

where Q is amount of drug release at time t, Q₀ is the initial amount of drug, Qₜ is the amount of drug remaining at time t, and where Qt/Q∞ is fraction of drug released at time t. k₀, k₁, and kₖₚ are the kinetic constants for zero order, first order, and Korsmeyer-Peppas models, respectively and n is the release exponent, indicative of the drug release mechanism. For Korsmeyer-Peppas model, only release data points were used in the analysis up to 60% drug release (2).

Results of our investigations:
Hausner factor determined by Stampfvolumeter: 1.173
Carr index: 15.28
Value of ASTM: 0.992 (according to the t_{g}=h/r function);
According to the results of the special and general mass-definition all values are corresponding to the Pharmacopoeia specifications;
In case of N=30 tablets the highest difference was 5% which can be maximum±10%

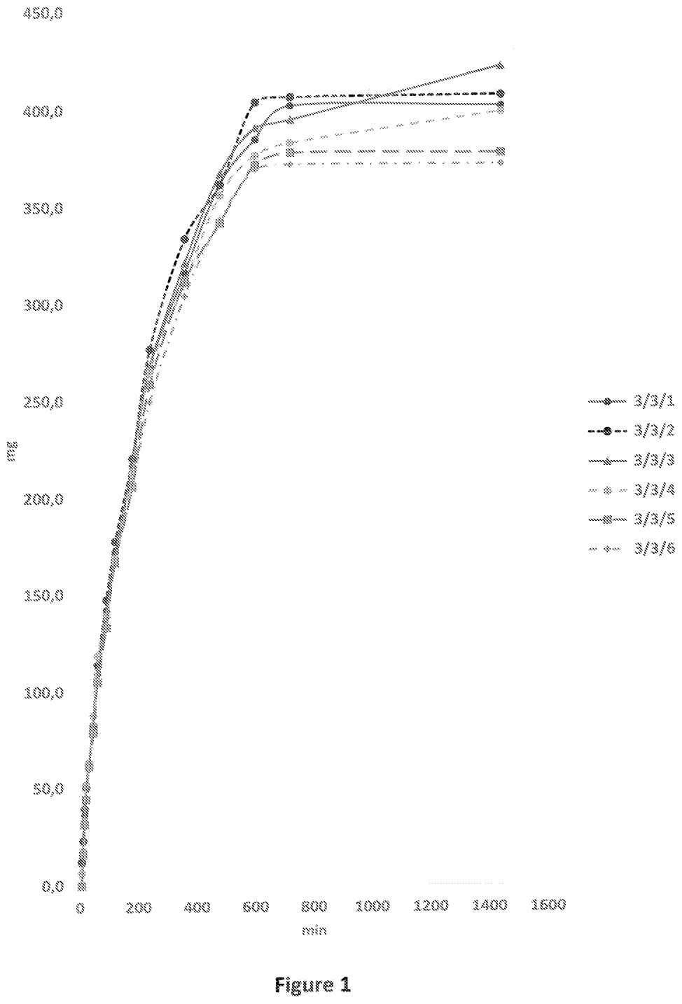

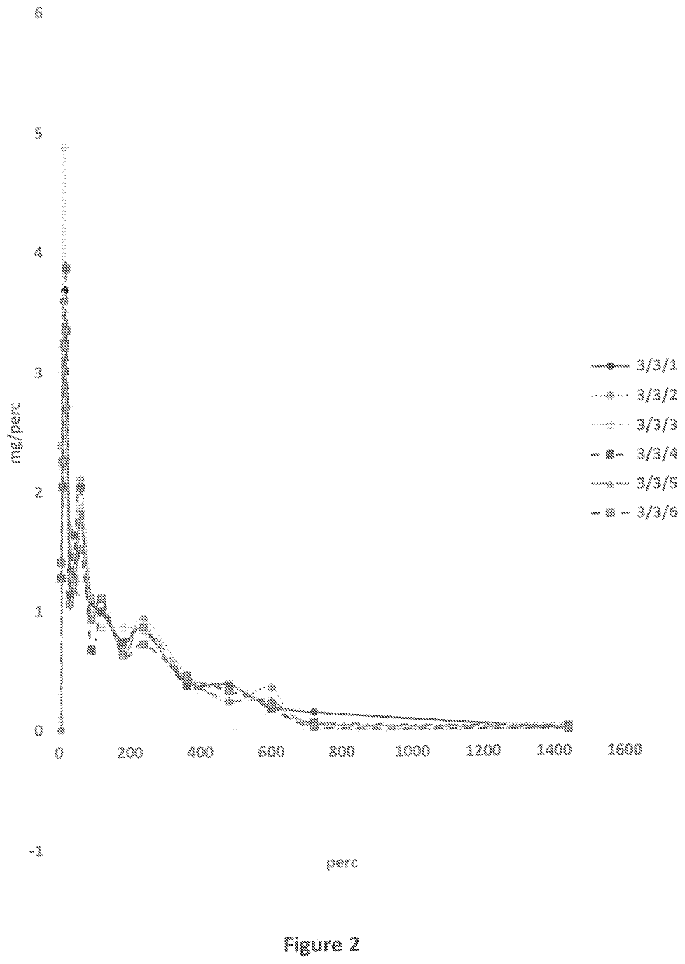

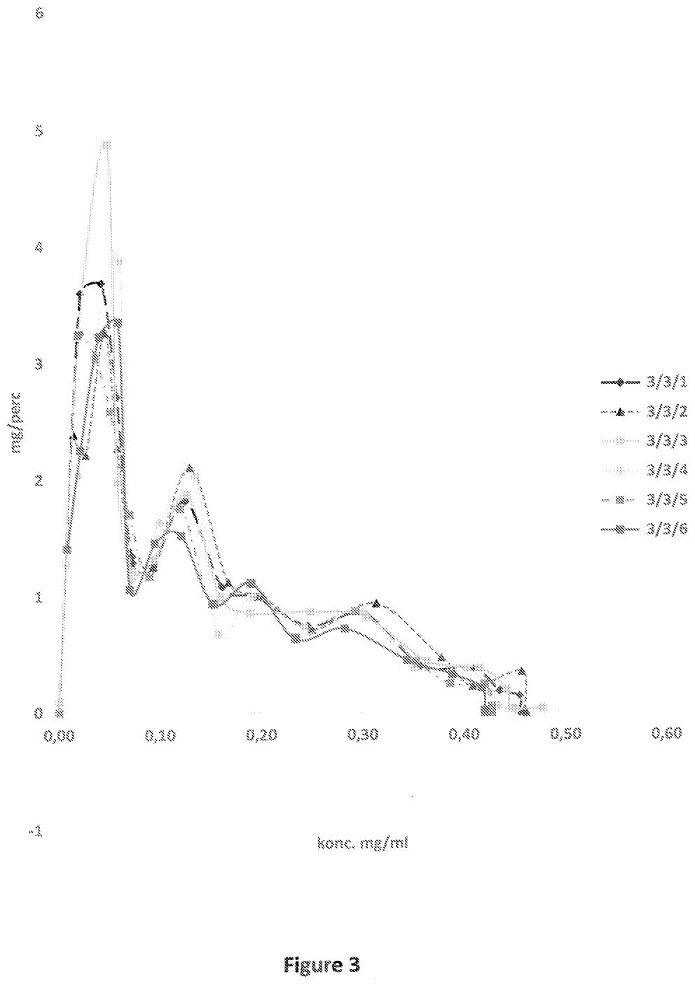

The kinetics of the active ingredient release is represented in FIG. 1, FIG. 2 and FIG. 3.

Based on the test results of the dissolution test, we can state that of 28.33% of the API dissolved in 1 hour. Additionally, 54.64% of the API was released in 3 hours. Finally, 80.83% of the API was released to the dissolution media in 6 hours. The result of the dissolution efficacy calculation was 86.40%. When the data of the two mostly varying samples were compared the result of the difference and similarity factor calculations were found to be 4.03 and 64.87, respectively. First order model was to be the best model describing the release of the API from the hydrophilic matrices, R²=0.9651 and in case of Korsmeyer-Peppas model the value was R²=0.9226.

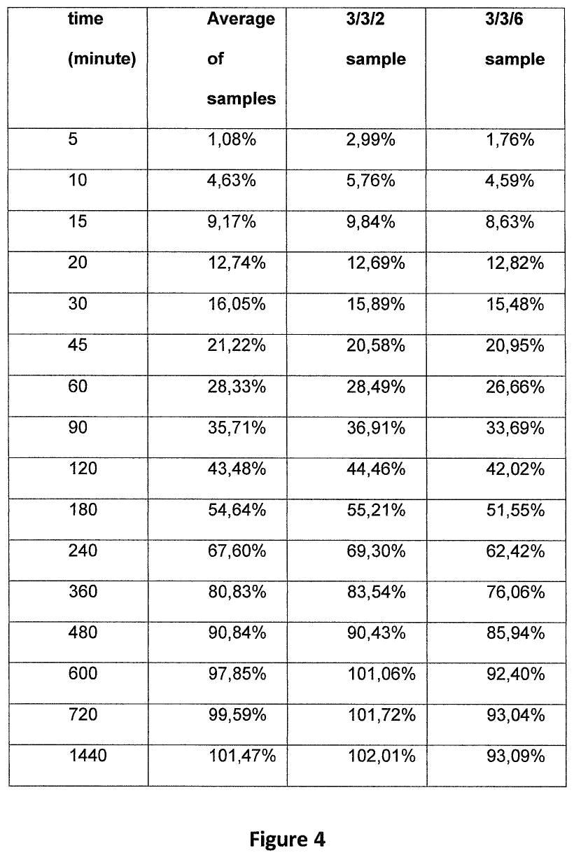

According to the measured rheological values the investigated granulate shows prime flow features and so the granulate is extremely well usable for formulation of a tablet. As it is shown on FIG. 4³⁷ and according to the release kinetics of the active ingredient of the composition the final conclusion is that the sustained and/or controlled release tablet according to the invention provides in vivo constant and continuous therapeutic concentration in case of 2×1 daily dosage. ³⁷1. Vasvari G, Csontos B, Sovany T, Regdon G, Jr., Benyei A, Varadi J, et al. Development and Characterisation of Modified Release Hard Gelatin Capsules, Based on In Situ Lipid Matrix Formation. AAPS Pharm Sci Tech. 2018. doi: 10.1208/s12249-018-1146-5. 2. Costa P, Manuel J, Lobo S. Modeling and comparison of dissolution profiles. Eur J Pharm Sci. 2001; 13(2):123-33. doi: Doi 10.1016/S0928-0987(01)00095

### SUMMARY

The present invention relates to a novel urinary, sustained and/or controlled-release alkalizing tablet for the oral treatment of interstitial cystitis/bladder pain syndrome (IC/BPS) and/or for general alkalization of the human body for long term where according to the subject matter of the invention the composition is potassium free, and comprises citric acid and citric acid salts as active ingredients.

As the active ingredient components are extremely hygroscopic a special formulation process was needed for formulating the alkalizing tablet using also different excipients with moisture relating capability.

Using the alkalizing tablet according to the invention following indications can be treated.
1) The alkalizing tablet is specially suitable for the oral treatment of the IC/BPS, because the pH value of the urine can provided for long term and continuously within the optimal neutral range of pH values between 6.9 and 7.5 advantageously on 7.38.
2) By using the sustained and/or controlled release composition according to the invention the required continuous neutral pH value of the urine is provided for 24 hours by 2×1 daily oral dosage where the active ingredient release during this period is nearly constant.
3) As the composition according to the invention is potassium-free, by using this potassium-free alkalizing tablet the pain and continuous micturition of the patient can be avoided. As it is well known from the literature., the potassium namely is strongly irritating the urethra and bladder of the IC/BPS patients
4) The extended-release alkalizing tablet is suitable for a general alkalization of the human body for long term. Because of the sustained and/or controlled release effect the alkalization of the body can be provided for long term.
   The advantages of the general alkalization are well known, but apart from our tablet according to the invention all usual alkalizing composition has a very short effect.

### REFERENCES

1 https://www.niddk.nih.gov/health-information/urologic-diseases/interstitial-cystitis-painful-bladder-syndrome/definition-facts
2 Vasudevan V., Moldwin R. Addressing quality of life in the patient with interstitial cystitis/bladder pain syndrome. Asian J Urol. 2017 January; 4(1):50-54.
3 Nickel J C, Tripp D A, Pontari M, Moldwin R, Mayer R, Carr L K, Doggweiler R, Yang C C, Nishra N, Nording J. Interstitial Cystitis/Painful Bladder Syndrome and Associated Medical Conditions With an Emphasis on Irritable Bowel Syndrome, Fibromyalgia and Chronic Fatigue Syndrome. The Journal of urology. 184.1358-63. 10.1016/j.juro.2010.06.005.
4 https://wvvw.niddk.nih.gov/health-information/urologic-diseases/interstitial-cystitis-painful-bladder-syndrome/treatment
5 Hanno P M, Wein A J, Malkowicz S B. Penn Clinical Manual of Urology 2017 217-34
⁶ Argade S, Chermansky C, Tyagi P. Biomarkers for Interstitial Cystitis/Painful Bladder Syndrome. Womens Health (Lond). 2016 January; 12(1): 87-90.
7 https://www.urologyhealth.org/urologic-conditions/interstitial-cystitis#Symptoms
8 https://www.ichelp.org/wp-content/uploads/2015/06/OLeary_Sant.pdf
9 Clemens J Q, Meenan R T, Rosetti M C, Gao S Y, Calhoun E A. Prevalence and incidence of interstitial cystitis in a managed care population. J Urol. 2005 January; 173(1):98-102
10 Suskind A M, Berry S H, Ewing B A, Elliott M N, Suttorp M J, Clemens J Q. The Prevalence and Overlap of Interstitial Cystitis/Bladder Pain Syndrome and Chronic Prostatitis/Chronic Pelvic Pain Syndrome in Men; Results of the RAND Interstitial Cystitis Epidemiology (RICE) Male Study. J Urol. 2013 January; 189(1); 141-145
11 Berry S H, Elliott M N, Suttorp M, Bogart L M, Stoto M A, Eggers P, Nyberg L, Clemens J Q. Prevalence of Symptoms of Bladder Pain Syndrome/Interstitial Cystitis Among Adult Females in the United States. J Urol 2011 August; 198(2); 540-544
12 Ibrahim I A, Diokno A C, Killinger K A, Carrico D J, Peters K M. Prevalence of self-reported interstitial cystitis (IC) and interstitial cystitis-like symptoms among adult women in the community. Int Urol Nephrol (2007) 39; 489-495
13 What is interstitial cystitis? 4 to 12 million may have IC. Interstitial Cystitis Association website, www.ichelp.org/about-ic/what-is-interstitial-cystitis/4-to-12-million-may-have-ic
¹⁴ https://www.urologyhealth.org/urologic-conditions/interstitial-cystitis
¹⁵ Tyagi P, Moon C H, Janicki J, Chancellor M, Yoshimura N, Chermansky C Recent advances in imaging and understanding interstitial cystitis. F1000Res. 2018; 7, Faculty Rev-1771
16 https://www.auanetorg/guidelines/interstitial-cystitis/bladder-pain-syndrome-(2011-amended-2014)
17 https://docplayer.net/20821777-Eating-with-ic-www-ichelp-org-interstitial-cystitis-association.html
18 https://www.ic-network.com/bev/
19 http://ic-diet.com/IC-diet-food-list.html
²⁰ Shorter B, Lesser M, Moldwin R M, Kushner L. Effect of comestibles on symptoms of interstitial cystitis. J Urol. 2007 July; 178(1): 145-52
²¹ Bassaly R, Downes K, Hart S. Dietary consumption triggers in interstitial cystitis/bladder pain syndrome patients. Female Pelvic Med Reconstr Surg. 2011
²² Ueda T, Yoshida T, Tanoue H, Ito M, Tamaki M, Ito Y, Yoshimura N. Urine alkalization improves the problems of pain and sleep in hypersensitive bladder syndrome. Int J Urol. 2014 May; 21(5):512-7
²³ Homma Y, Udea T, Tomoe H, Lin A T L, Kuo H C, Lee M H, Oh S J, Kim J C, Lee K S Clinical guidelines for interstitial cystitis and hypersensitive bladder, updated in 2015. Int J Urol. (2016) 23:542-549
²⁴ Nickel J Cl, Moldwin R, Lee S, Davis E L, Henry R A, Wyllie M G. Intravesical alkalinized lidocaine (PSD597) offers sustained relief from symptoms of interstitial cystitis and painful bladder syndrome. BJU Int. 2009 April; 103(7):910-8
²⁵ https://wvvw.vg.hu/kozelet/egeszsegugy-kozelet/osszeallitottak-a-leggyakrabban-hasznalt-gyogyszerek-listajat-1349833/26
²⁶ https://www.hazipatika.com/gyogyszerkereso/termek/milurit-300-mg-tabletta/1067
²⁷ https://www.drugs.com/condition/urinary-alkalinization.html
²⁸ Friedlander J I, Shorter B, Moldwin R M. Diet and its role in interstitial cystitis/bladder pain syndrome (IC/BPS) and comorbid conditions. BJU Int. 2012(109):1584-91
²⁹ Parsons C L. The role of a leaky epithelium and potassium in the generation of bladder symptoms in interstitial cystitis/overactive bladder, urethral syndrome, prostatitis and gynaecological chronic pelvic pain. BJU Int. 2010(107):370-5
³⁰ Montalbetti N, Stocker S D, Apodaca G, Bastacky S I, Carattino M D. Urinary K+ promotes irritative voiding symptoms and pain in the face of urothelial barrier dysfunction. www.nature.com/scientificreports
³¹ https://www.webmd.com/a-to-z-guides/hyperkalemia-potassium-importance#1
³² https://gibsociety.com/diet-icbps/
³³ https://www.drugs.com/cons/neut.html
³⁴ https://www.drugs.com/mtm/bicitra.html
³⁵ https://www.webmd.com/drugs/2/drug-6753/diamox-oral/details
³⁶ https://www.sciencedirect.com/topics/neuroscience/methazolamide

1. A medicinal and/or pharmaceutical urinary, extended-release alkalizing composition for the oral treatment of interstitial cystitis/bladder pain syndrome (IC/BPS) by alkalizing the urine and/or for general alkalization of the human body for long term, wherein the alkalizing composition is a sustained and/or controlled release and potassium-free composition, comprising:
the following components as active ingredients:
citric acid,
sodium citrate, and
magnesium citrate; and
the following components as excipients:
Aerosil,
Avicel DG,
Benecel hypromellose microcrystalline cellulose (HPMC) as grade; and
Magnesium stearate.

2. The medicinal and/or pharmaceutical urinary, extended-release alkalizing composition according to claim 1 wherein the composition is a tablet.

3. The medicinal and/or pharmaceutical urinary, extended-release alkalizing composition according to claim 1, wherein the composition is potassium-free.

4. The medicinal and/or pharmaceutical urinary, extended-release alkalizing composition according to claim 1, wherein the composition has a sustained and/or controlled release dosage form.

5. The medicinal and/or pharmaceutical urinary, extended-release alkalizing composition according claim 1, wherein the composition comprises the active ingredients with the following consistence:
50 to 100 mg citric acid and
130 to 250 mg sodium citrate and
190 to 290 mg magnesium citrate.

6. The medicinal and/or pharmaceutical urinary, extended-release alkalizing composition according to claim 5, wherein the composition comprises the active ingredients with the following consistence:
68 mg citric acid and
183 mg sodium citrate and
243 mg magnesium citrate.

7. The medicinal and/or pharmaceutical urinary, extended-release alkalizing composition according to claim 5, wherein the composition comprises the active ingredients with the following consistence:
53.17 mg citric acid and
143.09 mg sodium citrate and
190 mg magnesium citrate.

8. The medicinal and/or pharmaceutical urinary, extended-release alkalizing composition according to claim 1, wherein the composition comprises the excipients with the following consistence in weight percentage:
0.5% Aerosil;
15% to 25% Avicel DG;
15% to 25% Benecel hypromellose microcrystalline cellulose (HPMC) as grade; and
0.5% Magnesium stearate.

9. The medicinal and/or pharmaceutical urinary, extended-release alkalizing composition according to claim 8, wherein the consistence of the following excipients in weight percentage in the composition is the following:
19% Avicel DG; and
20% Benecel hypromellose microcrystalline cellulose (HPMC) as grade.

10. The medicinal and/or pharmaceutical urinary, extended-release alkalizing composition according to claim 1, wherein that the composition comprises Benecel K100M PH DC HPMC as grade.

11. The medicinal and/or pharmaceutical urinary, extended-release alkalizing composition according to claim 1, wherein a single administered dose of the composition achieves a therapeutic alkalizing concentration for providing a neutral pH value of the urine between values of 6.9 to 7.5 in an individual for about 8 to about 14 hours.

12. The medicinal and/or pharmaceutical urinary, extended-release alkalizing composition according to claim 11, wherein a single administered dose of the composition achieves a therapeutic alkalizing concentration for providing a neutral 7.38 pH value of the urine in an individual for 12 hours.

13. A process for the formulation of medicinal and/or pharmaceutical urinary, extended-release alkalizing composition according to claim in tablet form by the following steps for avoiding liquefaction and the formation of eutectic:
each active ingredient is pulverized separately until reaching the particle size of 6 fine-mesh sieve;
adding the excipient Aerosil with moisture retention capability to each active ingredient separately in small parts by continuous mixing in an amount of totally 0.5%;
providing the optimization of the rheological features of the granulates for formulating tablets by adding excipient Avicel DG with high moisture retention capability to each active ingredient separately in small parts by continuous mixing;
for providing the sustained and/or controlled release feature, adding Benecel K100M PH DC HPMC grade to each mixtures comprising each active ingredients separately and homogenizing each of the mixture of the components separately;
after combining of the three above prepared mixture of the excipients and ingredients and homogenizing the mixture, providing the required lubricant effect by adding excipient Magnesium stearate in an amount of 0.5%, and
formulating the granulates prepared by dry granulation into tablets by an excenter type Korsch tablet press machine.
